(19) United States
(12) Patent Application Publication
(10) Pub. No.: US 2013/0052276 A1
(43) Pub. Date: Feb. 28, 2013

(54) Method for Making an Antimicrobial Material from One-dimensional Nanometer Silver that Does Not Accumulate in a Human Body

(71) Applicants: Yi-Hsiuan Yu, Taoyuan County (TW); Ming-Hsiung Wei, Taoyuan County (TW); Lea-Hwung Leu, Taipei (TW); Kai-Yai Chang, Taipei (TW); Shen-fey Yeh, Taichung (TW); Fen-Yu Chung, Taichung (TW); Chen-Chi Ma, Hsinchu (TW)

(72) Inventors: Yi-Hsiuan Yu, Taoyuan County (TW); Ming-Hsiung Wei, Taoyuan County (TW); Lea-Hwung Leu, Taipei (TW); Kai-Yai Chang, Taipei (TW); Shen-fey Yeh, Taichung (TW); Fen-Yu Chung, Taichung (TW); Chen-Chi Ma, Hsinchu (TW)

(73) Assignee: Chung-Shan Institute of Science and Technology Armaments, Bureau, ministry of National Defence, Taoyuan County (TW)

(21) Appl. No.: 13/222,059

(22) Filed: Aug. 31, 2011

(51) Int. Cl. A61K 33/38; A61P 31/00

(52) U.S. Cl. 424/618

(57) ABSTRACT

Disclosed is a method for making an antimicrobial material from 1D nanometer silver that does not accumulate in a human body. At first, 1D nanometer silver is mixed in hydrophilic solution to produce 1D nanometer silver solution. Then, adhesive is blended in the 1D nanometer silver solution to produce the antimicrobial material. The antimicrobial material may be used in antimicrobial liquid, antimicrobial dressing or antimicrobial composite. Human skin can easily block the 1D nanometer silver. Therefore, the 1D nanometer silver does not enter or accumulate in the human body. Yet, the antimicrobial material exhibits a high bactericidal rate.

### BACKGROUND OF INVENTION

1. Field of Invention

The present invention relates to a method for making a silver-based antimicrobial material and, more particularly, to a method for making an antimicrobial material from one-dimensional (1 D) nanometer silver that can easily be blocked by the skin of a human body so that it cannot enter nor accumulate in the human body.

2. Related Prior Art

Silver can be used for bonding with the sulfur hydrogen bond of enzyme or protein on the cell walls of bacteria to prevent the cell walls from reproduction, to break the respiratory chains of the cells, to break the supply of energy, to prevent the bacteria from reproduction. Therefore, silver can be used to kill more than six hundred of bacteria such as fungi and spores. Silver exhibits antimicrobial activity against drug-resistant pathogenic bacteria such as Escherichia coli, staphylococcus aureus, bacterium pyocyaneum, streptococcus pyogene, entrococcus and anaerobium. Silver further exhibits excellent antimicrobial activity against staphylococcus aureus, caliform bacteria, bacterium pyocyaneum, candida albicans that are often found on the surface of a scald, burn or trauma. Silver further exhibits excellent antimicrobial chlamydia tracomatis, gonacoccus that causes tick-brone diseases. Silver can help with the healing of a wound. 100 silver ions are enough to kill bacteria. Silver is toxic to a limited extent. Argyria does not occur unless a human body contains more than 4 grams of silver. 6 grams of oral silver salt (including 3.8 grams of silver) seldom causes argyria.

When silver is provided in the order of a nanometer, it exhibits a large area of contact, and a very small amount of nanometer silver is enough to kill bacteria, and silver ions are released under control. The effect is lasting, and the cost is low. Silver does not give drug-resistance to bacteria but helps heal wounds. Silver exhibits several advantages. It does not require catalysis by light. It works in a wide range, lasts long, penetrates, and helps heal. Silver is non-toxic.

The present invention is therefore intended to obviate or at least alleviate the problems encountered in prior art.

### SUMMARY OF INVENTION

It is the primary objective of the present invention to provide a method for making an antimicrobial material from 1D nanometer silver that does not accumulate in a human body. Human skin can easily block the 1D nanometer silver. Therefore, the 1D nanometer silver does not enter or accumulate in the human body. Yet, the antimicrobial material exhibits a high bactericidal rate.

To achieve the foregoing objectives, the method includes the step of suspending 1D nanometer silver in hydrophilic solution to produce nanometer silver solution and the step of blending adhesive in the nanometer silver solution to produce the antimicrobial material.

The 1D nanometer silver may be in the form of filaments, tubes or rods.

The 1D nanometer silver may be made with a diameter of 10 nm to 100 nm and a length longer than 2 μm.

The hydrophilic solution is based on water, alcohol, polyhydric alcohol, lotion, gel and volatile spray.

The method may further include the step of blending additive in the 1D nanometer silver solution to provide a composite antimicrobial function.

The additive may be chitosan, absorptive carbon or moisturizer.

The antimicrobial material may be antimicrobial liquid.

Other objectives, advantages and features of the present invention will be apparent from the following description referring to the attached drawings.

### BRIEF DESCRIPTION OF DRAWINGS

The present invention will be described via detailed illustration of five embodiments referring to the drawings wherein:

FIG. 1 is a flow chart of a method for making a 1D nanometer silver solution;

FIG. 2 is a flow chart of a method for making an antimicrobial material from the 1D nanometer silver solution made in the method shown in FIG. 1 in accordance with the first embodiment of the present invention;

FIG. 3 is a flow chart of a method for making an antimicrobial material from the 1D nanometer silver solution made in the method shown in FIG. 1 in accordance with the second embodiment of the present invention;

FIG. 4 is a flow chart of a method for making a 1D nanometer silver solution;

FIG. 5 is a flow chart of a method for making an antimicrobial material from the 1D nanometer silver solution made in the method shown in FIG. 4 in accordance with the third embodiment of the present invention;

FIG. 6 is a flow chart of a method for making an antimicrobial material in accordance with the fourth embodiment of the present invention;

FIG. 7 is a flow chart of a method for making an antimicrobial material in accordance with the fifth embodiment of the present invention; and

FIG. 8 is a chart of the concentration of the nanometer in relation to the percent dissociation of silver ions in accordance with the present invention.

### DETAILED DESCRIPTION OF EMBODIMENTS

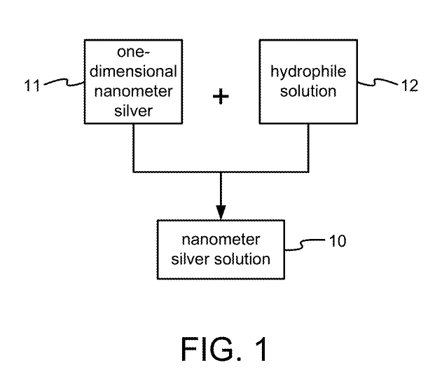

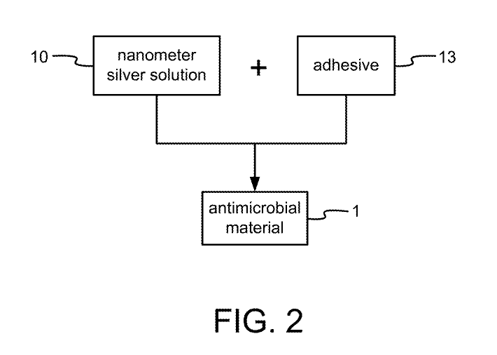

Referring to FIGS. 1 and 2, there is shown a method for making an antimicrobial material from 1D nanometer silver in accordance with a first embodiment of the present invention. The 1D nanometer silver does not accumulate in a human body.

Referring to FIG. 1, 1D nanometer silver 11 is mixed in hydrophilic solution 12, thus producing 1D nanometer silver solution 10. The 1D nanometer silver 11 may be in the form of filaments, tubes or rods. The diameter of the 1D nanometer silver 11 is 10 nm to 100 nm, and the length of the 1D nanometer silver 11 is longer than 2 μm. The hydrophilic solution 12 may be water, alcohol, polyhydric alcohol, lotion, gel or volatile spray.

Referring to FIG. 2, the 1D nanometer silver solution 10 is mixed with adhesive 13, thus producing an antimicrobial material 1. The antimicrobial material 1 may be antimicrobial liquid, dressing or composite. Such antimicrobial liquid may be antimicrobial spray, antimicrobial lotion, antimicrobial gel or antimicrobial cream. Such antimicrobial dressing may be carried on bandages, sanitary napkins or tampons. Such an antimicrobial composite may be a woven material, a filtering material, makeup, a toilet or personal wash.

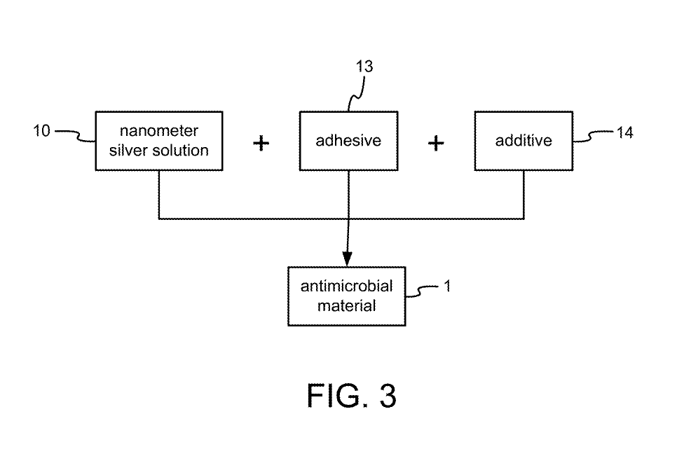

Referring to FIG. 3, there is shown a method for making an antimicrobial material from 1D nanometer silver in accordance with a second embodiment of the present invention. The second embodiment is like the first embodiment except mixing additive 14 with the 1D nanometer silver solution 10 and the adhesive 13 to provide the antimicrobial material 1 with a composite antimicrobial function. The additive 14 may be chitosan, absorptive carbon or moisturizer.

The length of the 1D nanometer silver 11 is longer than 10 micrometers. Hence, when the 1D nanometer silver 11 is used in various sanitary materials and dressing, the 1D nanometer silver 11 can easily be blocked by the skin of a human body. Therefore, the 1D nanometer silver 11 is kept out of the human body. That is, the 1D nanometer silver 11 does not enter or accumulate in the human body. On the other hand, the diameter of the 1D nanometer silver 11 is in the order of a nanometer so that the 1D nanometer silver 11 still possesses antimicrobial activity. Therefore, the antimicrobial material made from 1D nanometer silver in accordance with the present invention increases the values of the sanitary materials and contributes to the public sanitation while without jeopardizing the safety of the human body.

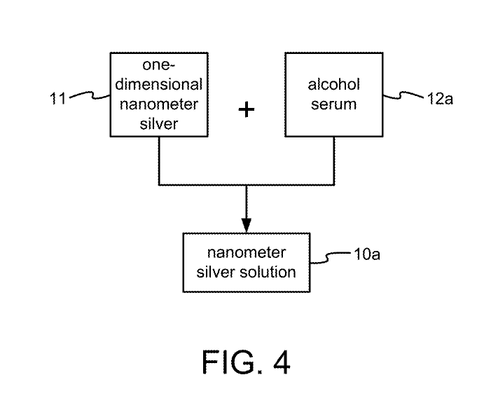

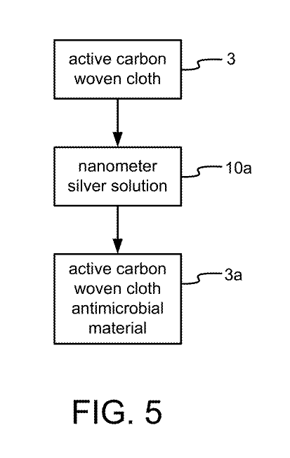

Referring to FIGS. 4 and 5, there is shown a method for making an antimicrobial material from 1D nanometer silver in accordance with a third embodiment of the present invention. At first, as shown in FIG. 4, 1 g of 1D nanometer silver 11 is mixed in 100 g of alcohol solution 12a to produce 1D nanometer silver solution 10a. The length of the 1D nanometer silver 11 is about 10 μm. The diameter of the 1D nanometer silver 11 is about 50 nm±20 nm.

Then, as shown in FIG. 5, 100 g of active carbon woven cloth 3 is soaked in the 1D nanometer silver solution 10a in an environment of negative pressure for about 1 hour. The active carbon woven cloth 3 soaked with the 1D nanometer silver solution 10a is heated at 80° C. in an oven. Thus, an active carbon woven cloth antimicrobial material 3a is provided with 1 wt % of silver.

The active carbon woven cloth antimicrobial material 3a was subjected to a quantitative analysis regulated by AATCC-100-2004 published by the American Association of Textile Chemists and Colorists versus blank active carbon woven cloth. The test was based on staphylococcus aureus. The test lasted for 24 hours. It was found that the amount of bacteria increased by 3 times on the blank active carbon woven cloth. Obviously, the blank active carbon woven cloth is not antimicrobial. On the other hand, substantially no bacterium was found on the active carbon woven cloth antimicrobial material 3a. Obviously, the active carbon woven cloth antimicrobial material 3a exhibits a bactericidal rate of 100%.

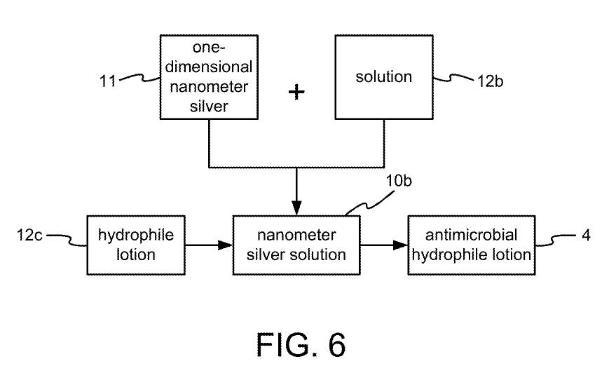

Referring to FIG. 6, there is shown a method for making an antimicrobial material from 1D nanometer silver in accordance with a fourth embodiment of the present invention. At first, 0.02 g of 1D nanometer silver 11 is mixed in 100 g of solution 12b to provide 1D nanometer silver solution 10b. The length of the 1D nanometer silver 11 is about 10 μm. The diameter of the 1D nanometer silver 11 is about 50 nm±20 nm. Then, 100 g of hydrophilic lotion 12c is added into the 1D nanometer silver solution 10b. The solution is stirred in a high speed homo mixer before it is subjected to vacuum to remove bubbles. Finally, there is provided antimicrobial hydrophilic lotion 4 with 0.01 wt % (100 ppm) of silver.

The antimicrobial hydrophilic lotion 4 was subjected to a test regulated by ASTM E2149 versus blank hydrophilic lotion. The test was based on staphylococcus aureus. The test lasted for 24 hours. It was found that the amount of bacteria increased by 3.5 times on the blank hydrophilic lotion. On the other hand, only a trace of bacterium was found in the antimicrobial hydrophilic lotion 4. Obviously, antimicrobial hydrophilic lotion 4 exhibits a bactericidal rate of 99.999%.

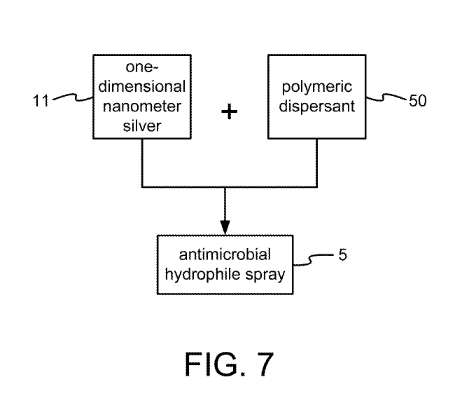

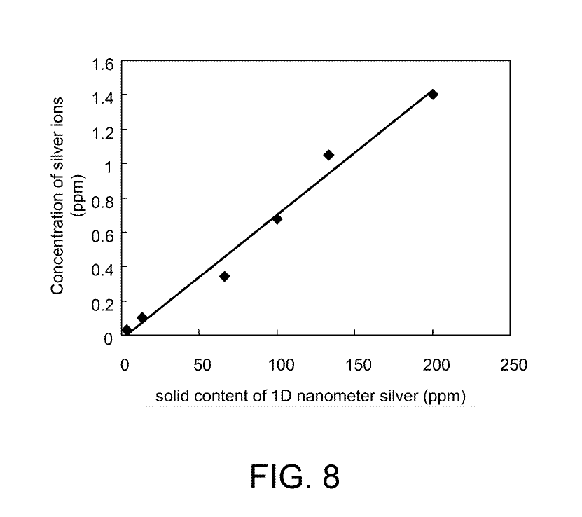

Referring to FIGS. 7 and 8, there is shown a method for making an antimicrobial material from 1D nanometer silver in accordance with a fifth embodiment of the present invention. At first, 0.02 g of 1D nanometer silver 11 and 0.01 g of polymeric dispersant (“PVP”) 50 are dissolved in 200 g of solution 12d to produce antimicrobial hydrophilic spray 5 with 0.01 wt % (100 ppm) of silver. The length of the 1D nanometer silver 11 is about 10 μm. The diameter of the 1D nanometer silver 11 is about 50 nm±20 nm.

The antimicrobial hydrophilic spray 5 was subjected to a test regulated by ASTM E2149 versus saline solution. The test was based on staphylococcus aureus. The test lasted for 24 hours. It was found that the amount of bacteria increased by 1.1 times in the saline solution. On the other hand, only a trace of bacterium was found in the antimicrobial hydrophilic spray 5. Obviously, the antimicrobial hydrophilic spray 5 exhibits a bactericidal rate of 99.7%. The concentration of the silver ions is 0.78 ppm.

Furthermore, 1D nanometer silver suspension with other concentration of silver ions were also tested. It was found that the solid content of the 1D nanometer silver is in a linear relation with the percent dissociation of silver ions as shown in FIG. 8.

As described in the foregoing embodiments, the method of the present invention exhibits the following advantages:

At first, the cost is low. A conventional antimicrobial material must include more than 3 wt % of silver to provide an adequate bactericidal rate. On the other hand, the 1D nanometer silver sanitary material of the present invention includes only 0.5 wt % of silver but provides an adequate bactericidal rate.

Secondly, there is no need for catalysis of the 1D nanometer silver by special light. The performance of the 1D nanometer silver of the present invention is better than that of titanium dioxide used as a photo-catalyst.

Thirdly, the nanometer silver kills more than 650 bacteria in minutes.

Fourthly, it lasts long. In an antimicrobial application, the nanometer silver is attached to the human skin. Hence, the nanometer silver can be mixed in various gels to release the silver ions under control. Only 1 ppm to 2 ppm of silver ions exhibits an excellent bactericidal rate.

Fifthly, it is safe for the human body. The length of the 1D nanometer silver filaments is longer than 10 micrometers so that the human skin can easily block the 1D nanometer silver filaments. That is, the 1D nanometer silver filaments do not enter or accumulate in the human body.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

1. A method for making an antimicrobial material from 1D nanometer silver that does not accumulate in a human body, the method includes the steps of:
suspending 1D nanometer silver in hydrophilic solution to produce 1D nanometer silver solution; and
blending adhesive in the 1D nanometer silver solution to produce the antimicrobial material.

2. The method for making an antimicrobial material from 1D nanometer silver that does not accumulate in a human body in accordance with claim 1, wherein the 1D nanometer silver is in a form selected from the group consisting of filaments, tubes and rods.

3. The method for making an antimicrobial material from 1D nanometer silver that does not accumulate in a human body in accordance with claim 1, wherein the 1D nanometer silver is made with a diameter of 10 nm to 100 nm and a length longer than 2 μm.

4. The method for making an antimicrobial material from 1D nanometer silver that does not accumulate in a human body in accordance with claim 1, wherein the hydrophilic solution is based on a material selected from the group consisting of water, alcohol, polyhydric alcohol, lotion, gel and volatile spray.

5. The method for making an antimicrobial material from 1D nanometer silver that does not accumulate in a human body in accordance with claim 4, further including the step of blending additive in the 1D nanometer silver solution to provide a composite antimicrobial function.

6. The method for making an antimicrobial material from 1D nanometer silver that does not accumulate in a human body in accordance with claim 5, wherein the additive is selected from the group consisting of chitosan, absorptive carbon and moisturizer.

7. The method for making an antimicrobial material from 1D nanometer silver that does not accumulate in a human body in accordance with claim 1, wherein the antimicrobial material is antimicrobial liquid.
